(19) United States
(12) Patent Application Publication
(10) Pub. No.: US 2019/0387904 A1
(43) Pub. Date: Dec. 26, 2019

(54) THERMAL RETENTION BLANKETS

(71) Applicant: Eugene Lloyd Hiebert, Salem, OR (US)

(72) Inventor: Eugene Lloyd Hiebert, Salem, OR (US)

(21) Appl. No.: 16/452,109

(22) Filed: Jun. 25, 2019

Related U.S. Application Data
(60) Provisional application No. 62/689,505, filed on Jun. 25, 2018.

(51) Int. Cl. A47G 9/02; A61F 7/00

(52) U.S. Cl. CPC A47G 9/0215; A61F 2007/0257; A61F 7/0097

(57) ABSTRACT

Exemplary blankets can comprise a flexible fluid-impermeable outer shell having two outer layers sealed around their perimeters, a flexible radiant barrier layer positioned inside the outer shell and being reflective of radiant energy, and a flexible thermal insulation layer positioned inside the outer shell adjacent the radiant barrier layer and comprising a material having low thermal conductivity, such that the blanket is capable of retaining patient body heat by creating a barrier to conductive, convective, and radiant patient body heat loss. Some disclosed blankets include an air cell layer or material capable of entrapping air, inside the outer shell, such as an exemplary open cell foam layer. Some disclosed blankets are MRI-compatible.

### CROSS-REFERENCE TO RELATED APPLICATIONS

This application claims the benefit of U.S. Provisional Patent Application No. 62/689,505, filed Jun. 25, 2018, which is incorporated by reference herein in its entirety.

### FIELD

This application is related to devices and methods for reducing heat loss in patients, such as during medical procedures.

### SUMMARY

The thermal retention blankets disclosed herein can help maintain normothermic body temperature, especially in situations where active or mechanical warming may not be available. The disclosed thermal retention blankets can be soft and supple, and can be draped or placed over and/or under the patient's body as the situation warrants to retain the most body heat. The thermal retention blankets can prevent patient body heat loss by radiation, conduction, and convection. The thermal retention blankets can be formed in various sizes and shapes dependent on the coverage needed due to the size and anatomy of the patient.

Exemplary blankets can comprise a flexible fluid-impermeable outer shell having two outer layers sealed around their perimeters, a flexible radiant barrier layer positioned inside the outer shell and being reflective of radiant energy, and a flexible thermal insulation layer positioned inside the outer shell adjacent the radiant barrier layer and comprising a material having low thermal conductivity and providing insulation against conduction of heat through the blanket, such that the blanket is capable of retaining patient body heat by creating a barrier to conductive, convective, and radiant patient body heat loss.

In some embodiments, an air cell layer or material capable of entrapping air (e.g., open cell foam layer) can be included to provide a layer of air within the outer shell for additional thermal insulation. In some embodiments, the radiant barrier layer can be made of an MRI-compatible material, or can be removed (e.g., replaced by the air cell layer), to provide a blanket that is MRI-compatible and can retain a patient's body heat during MRI scanning.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 is a plan view of an exemplary fluid impermeable external shell which comprises two outer layers, inside of which are a sheet of heat reflective layer or radiant barrier and a sheet of fibrous insulation material not shown in this figure. The two external fluid impermeable layers are sealed at their edges to form the shell. In the top left hand corner is a round grommet which is an example only and is not necessary for function. The grommet can be used to hang the blanket on a hook or attach it to something else.

FIG. 2 shows a sheet of fibrous insulation that is positioned inside the fluid impermeable external shell. This is a sample shape only.

FIG. 3 shows a sheet of heat reflective or radiant barrier, which is positioned inside the fluid impermeable external shell. The sheet of heat reflective layer or radiant barrier may be substituted for or supplemented with a sheet of an exemplary open cell foam or other material capable of entrapping air. This is a sample shape only.

FIG. 4 shows four layers of an exemplary thermal retention blanket. Two external fluid impermeable layers, one inner layer of fibrous insulation and one layer of heat reflective or radiant barrier or open an exemplary open cell foam sheet. This is a sample construction only.

FIG. 5 shows a human laying supine on a support structure which has a thermal retention blanket between the superior surface of the support structure and the posterior aspect of the human. The human has a second thermal retention blanket draped over its anterior and lateral aspects. This is illustrative only.

FIG. 6 shows a dog laying with its ventral surface on a support structure. Between the superior surface of the support structure and the ventral surface of the dog there is one thermal retention blanket. Draped over the dorsal and lateral aspects of the dog is another thermal retention blanket. This is illustrative only.

FIG. 7 is a graph that illustrates three phases of hypothermia.

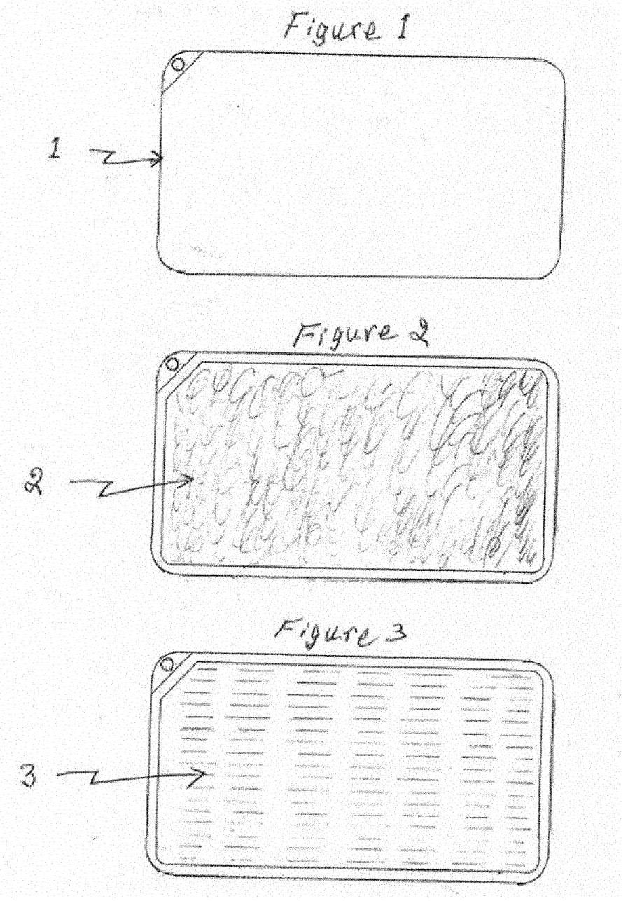

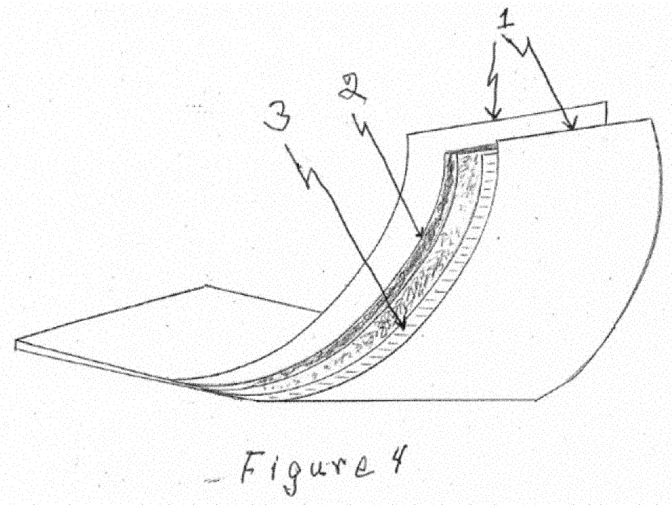

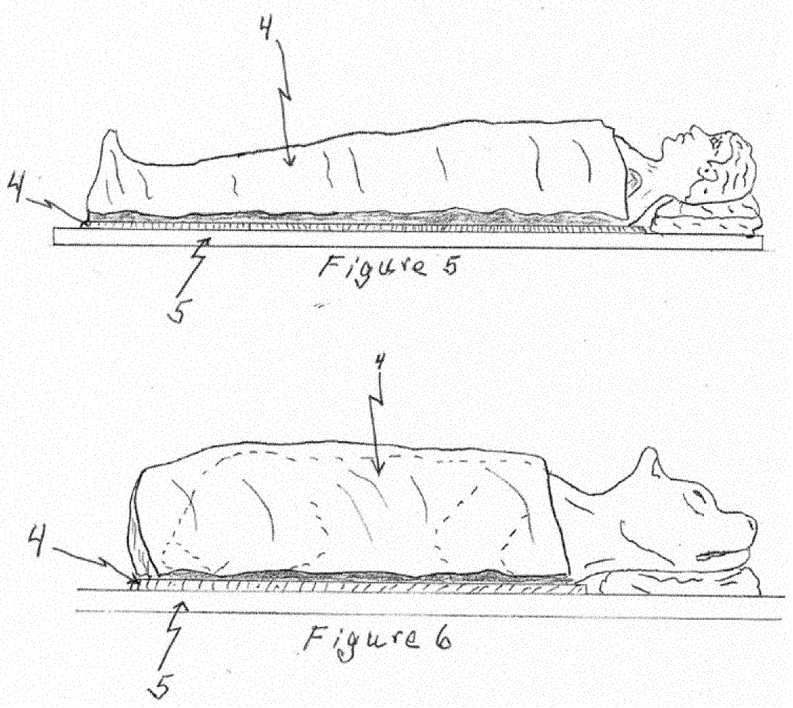

### DETAILED DESCRIPTION

Hypothermia is the most common thermal consequence of general anesthesia. Millions of humans and animals are anesthetized every year with anesthesia and surgical professionals struggling to maintain normothermia and the consequent deleterious physiological effects of hypothermia.

Hypothermia can be defined as the body temperatures below:

1) Human adults—below 35 degrees Centigrade

2) Human infants—below 36 degrees Centigrade

3) Dogs and cats—between 35.8 degrees Centigrade and 37 degrees Centigrade

The majority of patient heat loss during anesthesia and surgery is through the skin by the processes of radiation, conduction and convection as defined below.

1) Radiation:
Radiation is the major source of heat loss in surgical patients in which infrared radiant energy is transferred from the relatively warm patient to the environment.

2) Conduction:
Conduction refers to the direct flow of heat from the body to the surrounding air, fluids or solid materials such as a metal surgical table.

3) Convection:
Convection involves the physical movement of ambient air or fluids by which body heat is removed from the patient.

These three heat loss processes occur as core body heat redistributes to the periphery and the skin surface as a consequence of anesthetic induced peripheral vasodilation and depression of the hypothalamic thermoregulatory centers.

Hypothermia can occur in three phases following anesthetic induction:

### Phase 1:

In the first hour of anesthesia there is a rapid decline in body temperature as a consequence of anesthetic induced peripheral vasodilation and lowering of the temperature threshold in the hypothalamus preventing the institution of normal physiologic thermoregulatory mechanisms. These processes allow a redistribution of body heat from the body core to the periphery where heat is lost primarily through the skin by radiation and convection.

### Phase 2:

Over the next two hours of anesthesia, body temperature declines in a slower linear fashion as heat loss exceeds heat production. This occurs as a consequence of a decrease in metabolism and inhibition of heat production by thermoregulatory mechanisms in the hypothalamus by anesthetic drugs.

### Phase 3:

Over the next three to four hours of anesthesia a core body temperature plateau is reached after which temperature stabilizes and remains relatively unchanged as a thermal steady state is achieved.

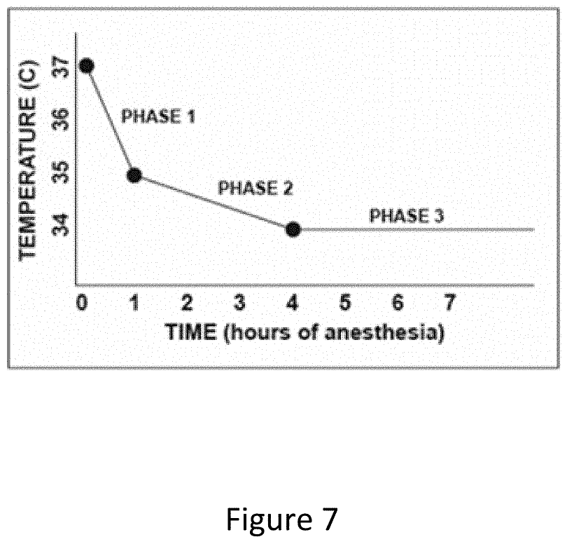

The graph in FIG. 7 is illustrative of these three phases of hypothermia.

Prolonged hypothermia can lead to significant morbidity and mortality causing health care professionals to maintain body temperature during anesthesia as normothermic as possible. Deleterious consequences of hypothermia can be as follows:
Cardiac arrhythmias
Increased peripheral vascular resistance (vasoconstriction)
Decreased oxygen uptake by red blood cells
Coagulopathy and platelet dysfunction
Postoperative protein catabolism and stress response
Altered mental status
Impaired renal function
Decreased drug metabolism
Poor wound healing
Increased surgical site infections
Death

### Current Warming Solutions:

Current solutions to hypothermia during anesthesia may be grouped into mechanical and nonmechanical methods. The mechanical methods include forced warm air from an electrical blower, electrical warming blankets and warm water circulating blankets which may be placed over and around the patient. Nonmechanical methods include regular blankets which have been warmed and “space blankets” which are essentially a sheet of aluminized foil to reflect body heat which are also placed over and around the patient. Most commercially available “space blankets” provide approximately 50 percent heat reflectivity and provide no barrier to conductive and convective body heat loss.

### Thermal Retention Blankets:

The thermal retention blankets disclosed herein can help maintain normothermic body temperature, especially in situations where active or mechanical warming may not be available. The disclosed thermal retention blankets can be soft and supple, and can be draped or placed over and/or under the patient's body as the situation warrants to retain the most body heat. The thermal retention blankets can prevent patient body heat loss by radiation, conduction, and convection. The thermal retention blankets can be formed in various sizes and shapes dependent on the coverage needed due to the size and anatomy of the patient. Some of these situations where patient body heat retention is needed can include, but not limited to, when:
only a portion of the body is available for covering with the thermal retention blanket because the remainder of the body is part of a sterile field or being operated upon.
the patient is anesthetized for short procedures and active or mechanical warming is unnecessary or complicates the performance of the procedure.
the patient is receiving MRI imaging where no ferrous metal, such as that contained in mechanical warming devices, can be in the imaging room and the patient requires anesthesia so that accurate imaging studies can be obtained.
the patient is receiving CT imaging and there is not sufficient space for active or mechanical warming or it may complicate the procedure.
the patient is receiving X-ray imaging and laying on a cold table and in a cold room without active warming.
the patient has a large surface area to mass ratio and is, therefore, especially susceptible to rapid heat loss. Examples include human infants and small animals.

In some embodiments, the disclosed thermal retention blankets can comprise at four or more layers, including:
The outer two fluid impermeable layers or shells are sealed at their edges to hermetically enclose the inner layers. These outer layers can comprise any material that is impermeable or nearly impermeable to fluid such as, but not limited to, polyvinylchloride sheet, urethane sheet, etc. The edges of these layers or shells can be sealed in any manner that might be compatible with their physical and molecular structure such as, but not limited to gluing, heat sealing, radio frequency welding, etc.
One of the inner layers can comprise a flexible heat reflective layer or radiant barrier layer, which can be made of Mylar or aluminum, for example. In some embodiment, it can have equal or near equal heat reflectivity on each side. The radiant barrier layer can occupy most (e.g., more than 50%, more than 75%, more than 90%, more than 95%, up to 100%) of the interior surface area of the thermal retention blanket. The radiant barrier layer can have a high level (e.g., more than 50%, more than 75%, more than 90%, more than 95%, up to 100%) of reflectivity of radiant heat. An exemplary radiant barrier layer was shown by ASTM C1371 testing to have 5 percent emissivity and 95 percent reflectivity of radiant heat. The radiant barrier layer may have thickness from 2 to 8 mil (0.002 to 0.008 inches), for example.
One of the inner layers can comprise a flexible sheet of thermal insulation material having a low thermal conductivity. This thermal insulation sheet can have a sufficient thickness (e.g., at least ½ inch thick, or at least 1 inch thick) to provide a barrier to thermal conductivity. This thermal insulation sheet can be made of a material such as, but not limited to, polyethylene, polypropylene, rayon, or other insulation material which has low thermal conductivity. This thermal insulation sheet can comprise a fibrous material or a non-fibrous material. This thermal insulation sheet can occupy most (e.g., more than 50%, more than 75%, more than 90%, more than 95%, up to 100%) of the interior surface area of the thermal retention blanket, and may or may not be bonded to the heat reflective layer or radiant barrier layer.

The thermal retention blankets can retain patient body heat by creating a barrier to conductive, convective, and radiant patient body heat loss in the following manners:

### Conductive Heat Loss:

The thermal retention blanket, by virtue of its thermal insulation layer, heat reflective layer or radiant barrier, and fluid impermeable outer shell, can form a thick insulating barrier to the direct conduction of heat from the body to the surrounding air or solid materials. In addition to being draped over the patient, it can also be placed underneath the patient to prevent conductive heat loss or direct transfer of heat to the underlying table or patient support surface.

### Convective Heat Loss:

The thermal retention blanket drapes over the patient to form a physical barrier between the patient and the circulating ambient air. Thereby body heat is trapped under the thermal retention blanket and is not transported away by flowing convection currents in the ambient atmosphere.

### Radiant Energy Loss:

The heat reflective layer or radiant barrier layer can be reflective of infra-red and other wavelengths of radiant energy on each side. In combination with the other layers, the heat reflective layer or radiant barrier layer is effective at substantially reducing radiant energy loss from the patient, reflecting most of the radiant energy from the patient back toward the patient. An exemplary radiant barrier layer has been shown by ASTM C1371 testing to reflect 95 percent of the radiant heat energy presented to it, mostly in the infra-red band.

Evaporative heat loss can also constitute a significant cause of heat loss in certain situations (e.g., with a wet or sweating patient). The disclosed thermal retention blanket can also prevent evaporative heat loss and loss of moisture from the body due to the fluid impermeable outer shell and the fluid impermeable radiant inner layer, which can also block water vapor and gases from passing through the blanket as well.

The color of the external fluid impermeable shell can be significant as well in the retention of patient body heat. The colors close to the color black will tend to absorb relatively more heat and colors close to the color white will tend to reflect relatively more heat, which is the more preferable color range in the disclosed blankets. For example, dull black has an emissivity value of 0.94 and a reflectivity value of 0.06. This indicates that the dull black color absorbs 94 percent of the heat or energy presented to it and only reflects 6 percent of the heat or energy presented to it. Conversely, a shiny chrome color would reflect virtually all the heat or energy presented to it and absorb very little heat or energy. Therefore, colors toward the lighter end of the color spectrum are preferable such as white, yellow, orange, and red. These colors in addition to a shiny surface would further enhance their reflectivity of energy and patient body heat and would be preferable in the external shell.

Exemplary components of the thermal retention blankets, including the fluid impermeable outer shell, the inner radiant reflective layer, and the inner fibrous insulating layer, have undergone individual specifications testing. The results of these tests are provided in U.S. Provisional Patent Application No. 62/689,505, filed Jun. 25, 2018, which is incorporated by reference herein in its entirety. An exemplary thermal retention blanket, as a whole, has undergone rigorous ASTM testing as well.

An entire exemplary thermal retention blanket was subjected to ASTM C1371 testing which measures the heat emissivity and heat reflectivity of the blanket. The emissivity of the surface of a material is its effectiveness in emitting energy as thermal radiation or its ability for energy or heat to leave an object. The reflectivity of a surface is its effectiveness in reflecting energy or heat as thermal radiation away from its surface. Reflectivity and emissivity are inversely related. The greater the reflectivity of a substance, the less the emissivity. The emissivity of the thermal retention blanket was 30 percent and the reflectivity was 70 percent. The heat reflectivity of the blanket, as a whole, was less than the heat reflectivity of the inner radiant reflective layer by itself (95 percent) because of the intervening fluid impermeable outer shell and the inner insulating layer. This test (ASTM C1371) measures only radiant energy reflectivity and does not measure the thermal resistance or transmittance of the combined action of conduction, convection and radiation.

An entire exemplary thermal retention blanket with four layers (two outer fluid impermeable layers, a heat reflective layer or radiant barrier, and a fibrous insulation layer) was tested in accordance with ASTM standard test ASTM D1518. This test determines the thermal resistance or transmittance of the combined layers or materials between a hot plate and the ambient atmosphere. This test provides an indication of the resistance to heat loss by conduction, convection and radiation. The result of this test is expressed as a CLO value. The higher the CLO value the greater the resistance to heat flow from the hot plate to the ambient atmosphere. Testing was performed on three specimens with the radiant heat reflective layer or radiant barrier face up from the hot plate and the fibrous insulation face up from the hot plate with the results below

| Radiant barrier face up | CLO Value |
|---|---|
| Specimen 1 | 3.64 |
| Specimen 2 | 2.68 |
| Specimen 3 | 3.46 |
| Average | 3.26 |
| Fibrous insulation face up | |
| Specimen 1 | 3.67 |
| Specimen 2 | 2.85 |
| Specimen 3 | 3.52 |
| Average | 3.35 |

The CLO value of an article of clothing or blanket is not familiar to the consuming public. The information below compares familiar clothing ensembles to their corresponding CLO values to provide a frame of reference.

| Clothing Ensemble | CLO Value |
|---|---|
| Nude | 0 |
| Shorts only | 0.1 |
| Light summer clothing | 0.5 |
| Typical indoor clothing | 1.0 |
| Heavy business suit | 1.5 |
| Business suit, overcoat | 2.0 |
| and hat | |
| Extreme cold weather | 3 to 4 |
| polar suit | |

The average CLO values of 3.26 for radiant barrier up and the CLO value of 3.35 for the fibrous insulation up indicate the effectiveness of the thermal retention blanket in providing a barrier to conductive, convective and radiant body heat loss. As can be seen the CLO value of Specimen 2 is somewhat of an outlier compared to Specimens 1 and 3 and therefore if one takes the average of the CLO values of Specimens 1 and 3 the average CLO value would be 3.6. The consistency of CLO values for Specimens 1 and 3 with either side up indicates that there is equal effectiveness to heat resistance with either side up. The CLO value of the herein disclosed thermal retention blankets can be at least 3.0 or even at least 3.5, as seen on the above chart, which can be equivalent to or superior to that of an extreme cold weather polar suit.

The thermal retention blanket, because of its barrier to radiant, conductive and convective heat losses, is most effective during Phase 1 of hypothermia (FIG. 7), such as after anesthetic induction when there is a rapid drop in body temperature due redistribution of warm core body blood to the periphery as a consequence of peripheral vasodilation by anesthetic medications. Body heat is consequently lost through the peripheral skin mainly through radiation and convection and to a lesser extent through conduction and evaporation. The thermal retention blanket acts as a barrier to heat loss through these heat loss processes. It is, therefore, imperative to prevent as much body heat loss as possible, such as by placing the thermal retention blanket on the patient prior to anesthetic induction or as soon as possible after anesthetic induction to prevent this initial rapid decrease in body heat during Phase 1.

The Thermal Retention Blanket with its internal radiant and insulative layers has been proven in clinical trials, when used immediately upon induction of anesthesia, to be effective in maintain patient normothermia even during long surgical procedures. One case report indicated normothermia even after a four hour surgical procedure. Clinical trials also showed that no signal loss or artifact was observed when used in X-ray and CT imaging. The Thermal Retention Blanket has been proven to be effective in maintaining normothermia in surgical and dental procedures as well as X-ray and CT imaging without image signal loss.

The thermal retention blanket with its internal reflective layer and internal insulative layer as has been described above may be used for maintaining body temperature in situations such as, but not limited to, surgery and imaging procedures including X-ray and CT (Computerized Axial Tomography) scanning. When used, however, with MRI (Magnetic Resonance Imaging) scanning the Radio Frequency signal from the MRI scanner may react with the internal heat reflective layer (when it contains certain MRI-incompatible materials, such as metal material) to produce isolated areas of elevated temperature in the thermal retention blanket. This is not desirable and the thermal retention blanket with an MRI-incompatible internal reflective layer is, therefore, not recommended in MRI scanning.

Some embodiments of the herein disclosed thermal retention blankets can be compatible with MRI and used during MRI scanning. In some such embodiments, the internal reflective layer or radiant barrier can be replaced with an internal layer of material that entraps air such as open cell foam sheet. In some embodiments, the open cell foam sheet can be about ⅛ inch to about 1 inch in thickness, such as about ¼ inch to about ½ inch in thickness. The open cell foam sheet can cover most of the interior surface of the shell, like other internal layers. The open cell foam sheet can provide an insulating layer of air to prevent heat loss in addition to the internal fibrous insulative layer as described above. The density of the open cell foam can be from 0.9 to 3.0 pounds per cubic foot. The Indentation Load Deflection (IDL) of the open cell foam, which is a measure of compression resistance can be from 10 to 40. The higher the IDL number the firmer the foam. The IDL of foam can range from 8 to 120, with 8 being very soft and essentially no compression resistance with 120 being very hard. The IDL of 10 to 40 is preferable so that the foam is soft and supple allowing the blanket to drape loosely over and around the patient and also provide a layer of entrapped air. The side of the thermal retention blanket with the open cell foam sheet can be next to the patient to create a layer of insulating air with the fibrous insulative layer above it to prevent heat loss from this layer of air. Open cell foam as opposed the closed cell foam is preferred because open cell foam is more supple and pliable compared to closed cell foam, making it superior for use in a blanket application. Additionally, in open cell foam there is an interconnected network of bubbles or air cells which have open walls allowing entrainment of air throughout the open cell matrix. Air is an excellent insulator and, therefore, the insulating value of the open cell foam relates to the amount of air inside the matrix of interconnected air cells with open walls. In some embodiments, closed cell foam or analogous non-foam materials that provide an air or gas layer can be used alternatively to closed cell foam.

The open cell foam sheet may be made of materials such as, but not limited to, polyurethane, polyethylene, polyester and polyamide. Since these materials do not interact with the Radio Frequency signal from the MRI scanner, they will not create areas of elevated temperature in the blanket and may be utilized in the blanket for use in MRI scanning. The thermal retention blanket designed with the open cell foam layer instead of the heat reflective layer or radiant barrier is not limited to MRI scanning and may be used in other clinical applications as well. In other embodiments, the foam layer can be replaced with any other material that is capable of trapping air.

Other materials that are capable of entrapping air would include natural fibers such as spun cotton or synthetic microfibers such as but not limited to polyester, olefin and polypropylene to name a few. These materials can be made of single types of microfibers such as 100 percent polyester which may come in weights from 60 grams per meter squared to 200 grams per meter squared and from ¼ inch to 1 inch thick with a Thermal Resistance measured in CLO value from 0.9 to 3. Spun polyester microfibers can be manufactured with olefin and polypropylene fibers in a 55 percent polyester and 45 percent olefin mixture as well as a 55 percent polyester and 45 percent polypropylene mixture. These microfiber fabric mixtures may be in weights from 40 grams per meter squared to 220 grams per meter squared, thickness from 0.15 inch to 1 inch and a Thermal Resistance expressed as a CLO value of 0.8 to 3.5. These materials can also be, but not limited to, 100 percent olefin or 100 percent polypropylene. These are exemplary air trapping materials only and do not limit this invention to the materials or combinations of these materials. Other materials for example may include polyethylene with air bubbles manufactured into the material to create a layer of air as an insulator (similar to “bubble wrap”).

The use of MRI scanning has become an essential imaging tool in human and veterinary medicine. Various metallic materials interfere with MRI imaging signals to effect imaging results and radio frequency signals from the MRI scanner can interact with various metallic materials to induce high temperatures in these materials. Consequently, no good methods of maintaining patient temperatures during long MRI scanning sessions have to this juncture been devised. Maintaining normothermia during MRI scanning sessions is essential to preventing morbidity and mortality during and after the MRI scanning session as has been previously described.

The disclosed thermal retention blankets designed for use with MRI have proven themselves to be effective maintaining normothermia and not interfering with MRI imaging signals in clinical trials. An exemplary thermal retention blanket was utilized as a clinical trial at an MRI center. It was found with multiple patients that normothermia was maintained even in scans over an hour in length and that there was no MRI signal loss despite using the thermal retention blanket in multiple different positions and there was also no inadvertent heating of the thermal retention blanket. This indicates that there is no undesirable interaction of the MRI signal with the components of the invention.

An exemplary thermal retention blanket compatible for MRI was also tested for inadvertent localized heating at an internationally recognized MRI compatibility testing laboratory with the following results. A single sample of a thermal retention blanket was tested for RF heating at both 1.5 T (64 MHz) and 3 T (128 MHz). A single sample was tested at both frequencies. A single heating test was conducted at each frequency (no repeats or variations were tested). All testing was generally conducted according to the relevant sections of ASTM F2182-11a:
RF exposure systems were used for 64 and 128 MHz exposure;
These systems were calibrated for RF power deposition (“Whole-body SAR”) using calorimetric methods described in F2182;
The blanket was placed over the top of an ASTM phantom (as defined in ASTM F2182-11 a) filled to a height of 9 cm with saline of conductivity verified to be between 0.45 and 0.5 S/m;
A finite number of fiber-optic temperature sensors were employed over the top surface of the blanket during exposure (7 in total); however, 7 fiber optic temperature probes were positioned at the corners, sides, and the middle of the blanket;
A thermal camera was used to obtain a measurement of the final temperature over the entire outer surface of the blanket following exposure at each frequency;
RF exposure was applied for a total of 20 minutes at each frequency.
   The results of testing were as follows:
Following 20 minutes of exposure to a whole-body SAR level of 2.99+/−0.15 W/kg at 64 MHz:
   peak temperature increases of not more than 0.2 deg-C. were observed at the fiberoptic temperature sensors. Given that the detection limit of the sensors used was 0.1 deg-C., this result is consistent with the conclusion that no heating of the blanket could be detected at 64 MHz
   The thermal camera data indicate no localized heating or hot-spots.
   Thermal photographs were taken of the device under test with 64 MHz, and the thermal camera images (before and immediately after RF exposure). No significant heating could be detected.
Following 20 minutes of exposure to a whole-body SAR level of 2.73+/−0.15 W/kg at 128 MHz:
   peak temperature increases of not more than 0.6 deg-C. were observed at the fiberoptic temperature sensors. Given that the detection limit of the sensors used was 0.1 deg-C., this result is consistent with the conclusion that very little heating of the blanket could be detected at 128 MHz
   The thermal camera data indicate no localized heating or hot-spots.
   Thermographic photographs of the device under test with 128 MHz, and the thermal camera images (before and immediately after RF exposure). No significant heating could be detected.

These results indicate that the components of the thermal retention blanket configured for MRI do not interact with MRI signals to produce unwanted heating of the components of the Blanket.

For purposes of this description, certain aspects, advantages, and novel features of the embodiments of this disclosure are described herein. The disclosed apparatuses, systems, and methods should not be construed as limiting in any way. Instead, the present disclosure is directed toward all novel and nonobvious features and aspects of the various disclosed embodiments, alone and in various combinations and sub-combinations with one another. The disclosed embodiments are not limited to any specific aspect or feature or combination thereof, nor do the disclosed embodiments require that any one or more specific advantages be present or problems be solved.

As used herein, the term “and/or” used between the last two of a list of elements means any one or more of the listed elements. For example, the phrase “A, B, and/or C” means “A,” “B,” “C,” “A and B,” “A and C,” “B and C” or “A, B and C.” As used herein, the term “coupled” generally means mechanically, chemically, electrically, magnetically or otherwise coupled or linked and does not exclude the presence of intermediate elements between the coupled items, unless otherwise described herein.

In view of the many possible embodiments to which the principles disclosed herein may be applied, it should be recognized that the illustrated embodiments are only examples and should not be taken as limiting the scope of the disclosure. Rather, the scope of the disclosure is at least as broad as the following claims. I therefore claim all that comes within the scope and spirit of these claims.

1. A thermal retention blanket, comprising:
a flexible fluid-impermeable outer shell having two outer layers sealed around their perimeters;
a flexible radiant barrier layer positioned inside the outer shell, the radiant barrier layer being reflective of radiant energy; and
a flexible thermal insulation layer positioned inside the outer shell adjacent the radiant barrier layer, the thermal insulation layer comprising a material having low thermal conductivity and providing insulation against conduction of heat through the blanket;
wherein the blanket is configured to retain patient body heat by creating a barrier to conductive, convective, and radiant patient body heat loss.

2. The blanket of claim 1, wherein the blanket has a CLO value greater than 3.0.

3. The blanket of claim 1, wherein the blanket has a CLO value greater than 3.5.

4. The blanket of claim 1, wherein the radiant barrier layer has a reflectivity of at least 75%.

5. The blanket of claim 1, wherein the radiant barrier layer has a reflectivity of at least 90%.

6. The blanket of claim 1, wherein the radiant barrier layer has a reflectivity of at least 95%.

7. The blanket of claim 1, wherein the radiant barrier layer comprises aluminum or Mylar.

8. The blanket of claim 1, wherein the thermal insulation layer comprises a fibrous material.

9. The blanket of claim 1, wherein the thermal insulation layer has a thickness of at least 1 inch.

10. The blanket of claim 1, wherein the radiant barrier layer is MRI compatible.

11. The blanket of claim 1, further comprising an open cell foam layer positioned inside the outer shell.

12. An MRI-compatible thermal retention blanket, comprising:
a flexible fluid-impermeable outer shell having two outer layers sealed around their perimeters;
a flexible air cell layer positioned inside the outer shell, the air cell layer comprising a plurality of cells containing air and configured to create a heat insulating layer of air within the outer shell to prevent heat loss; and
a flexible thermal insulation layer positioned inside the outer shell adjacent the radiant barrier layer, the thermal insulation layer comprising a fibrous material having low thermal conductivity and providing insulation against conduction of heat through the blanket;
wherein the blanket is configured to retain patient body heat during MRI scanning by creating a barrier to heat loss.

13. The blanket of claim 12, wherein the air cell layer comprises a foam layer.

14. The blanket of claim 12, wherein the air cell layer comprises an open cell foam layer.

15. The blanket of claim 14, wherein the open cell foam layer has a thickness in a range of ¼ inch to ½ inch.

16. The blanket of claim 12, wherein the thermal insulation layer comprises a fibrous material.

17. The blanket of claim 12, wherein the air cell layer comprises a closed cell layer comprising a plurality of closed cells that contain air.

18. The blanket of claim 12, wherein the blanket has a CLO value greater than 3.0.

19. The blanket of claim 12, wherein the blanket has a CLO value greater than 3.5.

20. The blanket of claim 12, further comprising flexible radiant barrier layer positioned inside the outer shell, the radiant barrier layer being at least 95% reflective of radiant energy.
